(19) United States
(12) Patent Application Publication
(10) Pub. No.: US 2026/0062804 A1
(43) Pub. Date: Mar. 5, 2026

(54) METHOD OF TREATING THIN FILMS AND METHOD OF MANUFACTURING MEMORY DEVICE

(71) Applicant: EGTM Co., Ltd., Suwon-si (KR)

(72) Inventors: Kyu Ho CHO, Suwon-si (KR); Hyun Ju JUNG, Suwon-si (KR); Ha Na KIM, Suwon-si (KR); Ju Hwan JEONG, Suwon-si (KR); Hyeon Sik CHO, Suwon-si (KR); Han Bin LEE, Suwon-si (KR); Sun Young BAIK, Suwon-si (KR); Ji Yeon HAN, Suwon-si (KR)

(73) Assignee: EGTM Co., Ltd., Suwon-si (KR)

(21) Appl. No.: 19/316,956

(22) Filed: Sep. 2, 2025

(30) Foreign Application Priority Data
Sep. 2, 2024 (KR) 10-2024-0118878

(51) Int. Cl. C23C 16/455; C07C 43/12; C07C 43/32; C09K 13/00; C23C 16/40; C23C 16/44

(52) U.S. Cl. CPC C23C 16/45527; C07C 43/12; C07C 43/32; C09K 13/00; C23C 16/405; C23C 16/4408

(57) ABSTRACT

Disclosed is a method of treating thin films, the method comprising: supplying a capping precursor to the inside of a chamber where a substrate is placed to adsorb the capping precursor onto a thin film formed on the substrate; purging the inside of the chamber; supplying a first reaction material to the inside of the chamber to form a stressor layer; purging the inside of the chamber; annealing the substrate; supplying an etch initiator to the inside of the chamber; purging the inside of the chamber; supplying a second reaction material to the inside of the chamber to activate the etch initiator, and purging the inside of the chamber.

### TECHNICAL FIELD

The present invention relates to a method of treating thin films and a method of manufacturing a memory device including the same, and more particularly, to a method of treating thin films using capping via a stressor layer for improving thin film properties, and a method of manufacturing a memory device including the same.

### BACKGROUND

With advances in integrated circuit electronic devices, improving device performance and miniaturization have become important issues. Particularly, in metal-oxide-semiconductor (MOS) devices, the performance of the gate dielectric greatly affects overall device characteristics. However, as the thickness of conventional silicon dioxide (SiO₂) dielectrics is reduced, leakage current increases, lowering device reliability.

To address this, high-k materials are used as dielectrics. High-k materials maintain a high dielectric constant even at low thickness, improving electrical performance and effectively lowering leakage current. Thus, even miniaturized devices can maintain high performance and reliability.

However, as the thickness of high-k materials is reduced, defects or impurities can make it difficult to achieve the desired properties. Thin high-k dielectric films are more sensitive to such imperfections, which may greatly affect overall electrical properties. The thinner the films, the more significant are surface/interface effects. Therefore, obtaining high-quality, thin high-k dielectrics is a crucial technical challenge.

As a result, development of high-k materials capable of maintaining high quality even at low thickness is actively being pursued to maximize performance and reliability of low-power logic devices, non-volatile memories, capacitors, and other high-k metal oxide-based semiconductor devices.

An object of the present invention is to provide a method of treating thin films and a method of manufacturing a memory device including the same, which can improve thin film properties by using capping through a stressor layer.

Other objects of the present invention will become more apparent from the following detailed description.

### SUMMARY

Disclosed is a method of treating thin films, the method comprising: supplying a capping precursor to the inside of a chamber where a substrate is placed to adsorb the capping precursor onto a thin film formed on the substrate; purging the inside of the chamber; supplying a first reaction material to the inside of the chamber to form a stressor layer; purging the inside of the chamber; annealing the substrate; supplying an etch initiator to the inside of the chamber; purging the inside of the chamber; supplying a second reaction material to the inside of the chamber to activate the etch initiator; and purging the inside of the chamber.

Disclosed is a method of treating thin films, the method comprising: forming first and second stressor layers on a thin film formed on a substrate, alternately and repeatedly; annealing the substrate; supplying an etch initiator to the inside of the chamber where the substrate is placed; purging the inside of the chamber; supplying a third reaction material to the inside of the chamber to activate the etch initiator; and purging the inside of the chamber, wherein forming the first stressor layer comprising: supplying a first capping precursor to the inside of a chamber to adsorb the first capping precursor onto the thin film; purging the inside of the chamber; supplying a first reaction material to the inside of the chamber to form a first stressor layer; purging the inside of the chamber, wherein forming the second stressor layer comprising: supplying a second capping precursor to the inside of a chamber to adsorb the second capping precursor onto the thin film; purging the inside of the chamber; supplying a second reaction material to the inside of the chamber to form a second stressor layer; purging the inside of the chamber.

The etching initiator may be represented by the following <Chemical Formula 1>:

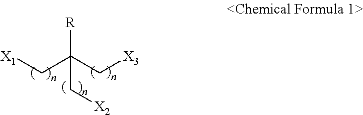

in <Chemical Formula 1>, n is each independently selected from integers of 0 to 5,
X1 to X3 are each independently selected from an alkoxy group having 1 to 5 carbon atoms, and a dialkylamino group having 1 to 5 carbon atoms, and
R is selected from hydrogen, a linear, branched, or cyclic alkyl groups having 1 to 5 carbon atoms, an alkoxy group having 1 to 5 carbon atoms, and a dialkylamino group having 1 to 5 carbon atoms.

The etching initiator may be any one of Trimethyl orthoformate (TMOF), Triethyl orthoformate (TEOF), Dimethylformamide dimethyl acetal (DFDA), and Tris(dimethylamino)methane (TDMAM).

The etching initiator may be represented by the following <Chemical Formula 2> or <Chemical Formula 3>:

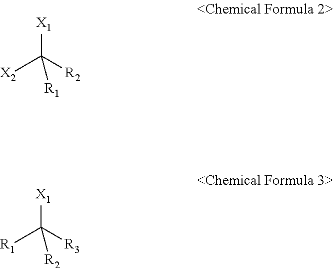

in <Chemical Formula 2> or <Chemical Formula 3>, X1 to X2 are independently hydrogen, chlorine element, and a chloroalkyl group having 1 to 5 carbon atoms,
R1 to R3 are independently selected from hydrogen, linear, branched, or cyclic alkyl groups having 1 to 5 carbon atoms, aryl groups having 6 to 12 carbon atoms, hydroxy groups having 0 to 4 carbon atoms, and alkoxy groups having 0 to 4 carbon atoms.

The etching initiator may be any one of dichloromethyl methyl ether (DCMME), trimethyl chloro orthoacetate (TMCOA), and chloromethyl ethyl ether (CMEE).

The thin film may be a metal oxide film having one of Al, Nb, Hf, Ti, Si, Ta, Mo, Zr, and W as a central element.

The stressor layer may be a metal oxide film having one of Nb, Ta, Cr, Zr, Ru, Mo, and Sn as a central element.

The stressor layer may have a band gap smaller than that of the thin film.

The stressor layer may have a higher crystallization temperature than the thin film.

The reactant may be any one of O₃, O₂, or H₂O.

The method may be performed at 50 to 700° C.

The thin film and the stressor layer may be any one of a metal film, a metal oxide, a metal nitride, a metal sulfide, a silicon nitride, and a silicon oxide.

The thin film may be a binary compound or a ternary compound doped with one or more elements.

Disclosed is a method of manufacturing a memory device may include the aforementioned method of treating thin films.

### BRIEF DESCRIPTION OF THE DRAWINGS

FIG. 1 is a diagram illustrating annealing results with and without the stressor layer.

FIG. 2 is a diagram illustrating annealing/etching results according to the property of the thin film.

FIG. 3 is a flowchart illustrating a method of treating thin films according to an embodiment of the present invention.

FIG. 4 is a graph schematically showing a supply cycle according to an embodiment of the present invention.

FIG. 5 is a diagram illustrating annealing/etching according to an embodiment of the present invention.

FIG. 6 is XRD results of HfO₂ for Comparative Example 1/Example 1.

FIG. 7 is TEM photos showing annealing/etching results according to Example 1.

FIG. 8 is XRD results of HfO₂ for Comparative Example 2/Example 2.

FIG. 9 is a flowchart illustrating a method of treating thin films according to another embodiment of the present invention.

FIG. 10 is a graph schematically showing a supply cycle according to another embodiment of the present invention.

FIGS. 11 and 12 are diagrams of annealing/etching according to an embodiment of the present invention.

FIG. 13 is a diagram illustrating Comparative Examples 3-1 to 3-6, and Example 3.

FIG. 14 is a table showing the electrical properties according to Comparative Examples 3-1 to 3-6, and Example 3.

FIG. 15 is a graph showing the electrical properties according to Comparative Examples 3-1 to 3-6, and Example 3.

FIG. 16 is a graph showing P-E curve for the thin film of Comparative Example 4.

FIG. 17 is a graph showing P-E curve for the thin film of Example 4.

FIG. 18 is a table showing the electrical properties according to Comparative Example 4/Example 4.

### DETAILED DESCRIPTION

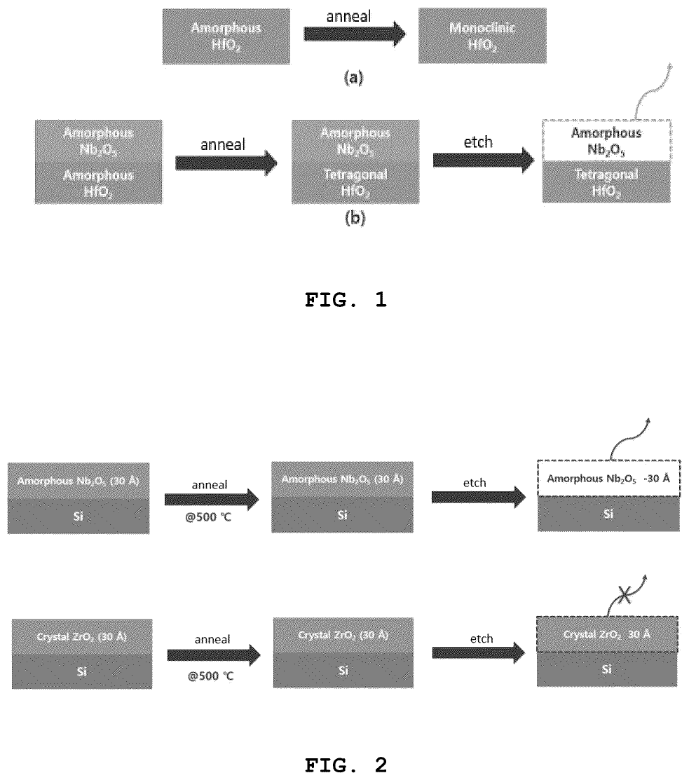

Hereinafter, preferred embodiments of the present invention will be described in detail with reference to FIGS. 1 to 18. It should be understood that the embodiments of the present invention can be modified in various forms, and the scope of the present invention should not be construed as being limited to the embodiments described below. These embodiments are provided to more fully explain the invention to those skilled in the art. Thus, the shapes of respective elements shown in the drawings may be exaggerated in order to emphasize more distinct explanation.

FIG. 1 is a diagram illustrating annealing results with and without the stressor layer, FIG. 2 is a diagram illustrating annealing/etching results according to the property of the thin film.

By capping the upper part of a metal oxide layer with a metal oxide layer of another element, stress is applied at the interface, and in this case, the lower thin film can form a stable crystal structure so as to minimize the stress. As a result, crystallinity of the lower thin film can be improved or its crystallization temperature can be lowered. In addition, the metal oxide used as the stressor layer has a high crystallization temperature, and thus, after capping and annealing, even if the lower film crystallizes, the stressor layer remains in an amorphous structure. Therefore, even after the lower film is crystallized, the amorphous stressor layer smooths the interface, improving the surface roughness of the lower thin film.

However, since this stressor layer can also be a cause of deterioration of the properties of the lower thin film, an etching process for removing the stressor layer is required to prevent unwanted property deterioration. The amorphous structure of the stressor layer has a high surface energy due to its irregular arrangement, which leads to high chemical reactivity and makes it easy to etch. However, the lower thin film, with its relatively regular arrangement and crystalline structure, has low chemical reactivity, making it difficult to etch, and thus it can serve as an etch stop layer, which is advantageous for controlling etching process. Additionally, the metal oxide used as the stressor layer is made of a material with a smaller band gap than the lower thin film, and in this case, the higher chemical reactivity enables easier etching.

According to the above-described method, a dielectric film having improved stability and high dielectric constant at low thickness can be provided. Such a thin film structure can be used in various electronic devices such as transistors, capacitors, and integrated circuit devices, and can improve the characteristics of such electronic devices.

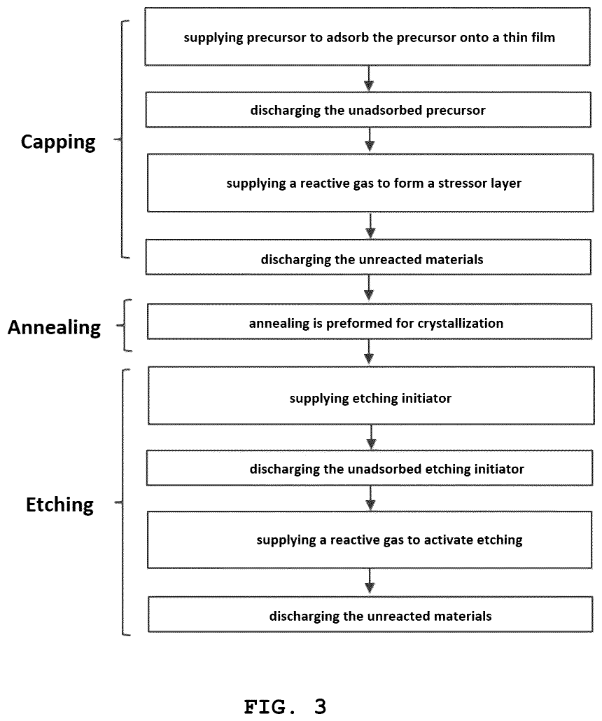

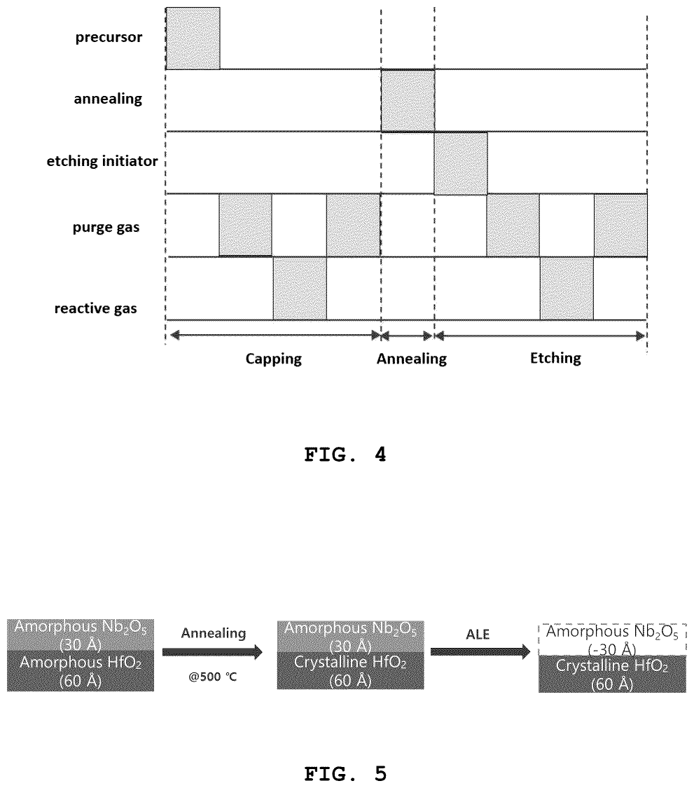

FIG. 3 is a flowchart illustrating a method of treating thin films according to an embodiment of the present invention, FIG. 4 is a graph schematically showing a supply cycle according to an embodiment of the present invention. A substrate is loaded into a process chamber, and the substrate has a thin film formed on its surface. The thin film may have Hf, Zr, Al, Ta, or Ti as its central element, and may be any one of a metal film, a metal oxide, a metal nitride, a metal sulfide, a silicon nitride, or a silicon oxide. The thin film may be a binary compound or ternary compound doped with one or more elements.

Meanwhile, the following process conditions can be adjusted. The process conditions may include a temperature of the substrate or process chamber, a pressure in the process chamber, gas flow rate, and the temperature is 50 to 700° C.

The substrate is exposed to a capping precursor supplied to the inside of the chamber, and the capping precursor is adsorbed on the thin film formed on the surface of the substrate. The capping precursor may have Nb, Ta, Cr, Zr, Ru, Mo, or Sn as its central element.

Thereafter, a purge gas (for example, an inert gas such as Ar) is supplied to the inside of the chamber to discharge the unadsorbed precursor or by-products.

Thereafter, the substrate is exposed to a reactant (or reactive gas) supplied to the inside of the chamber, forming a stressor layer by the reactant. The reactant may be any one of O₃, O₂, or H₂O. The stress layer may have one of Nb, Ta, Cr, Zr, Ru, Mo, and Sn as a central element. The stressor layer may have a band gap smaller than that of the thin film, and a higher crystallization temperature than the thin film.

Thereafter, a purge gas (for example, an inert gas such as Ar) is supplied to the inside of the chamber to discharge the unreacted materials or by-products.

Thereafter, annealing is performed on the substrate, which may be conducted at 50 to 700° C. with a reaction material (e.g., O₂ atmosphere) supplied (see FIG. 5).

The substrate is exposed to an etching initiator supplied to the inside of the chamber. The etching initiator is supplied at 50 to 700° C.

Specifically, the etching initiator may be represented by the following <Chemical Formula 1>

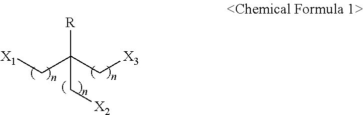

in <Chemical Formula 1>, n is each independently selected from integers of 0 to 5,
X1 to X3 are each independently selected from an alkoxy group having 1 to 5 carbon atoms, and a dialkylamino group having 1 to 5 carbon atoms, and
R is selected from hydrogen, a linear, branched, or cyclic alkyl groups having 1 to 5 carbon atoms, an alkoxy group having 1 to 5 carbon atoms, and a dialkylamino group having 1 to 5 carbon atoms.

Specifically, the etching initiator may be any one of Trimethyl orthoformate (TMOF), Triethyl orthoformate (TEOF), Dimethylformamide dimethyl acetal (DFDA), and Tris(dimethylamino)methane (TDMAM).

Also, the etching initiator may be represented by the following <Chemical Formula 2> or <Chemical Formula 3>:

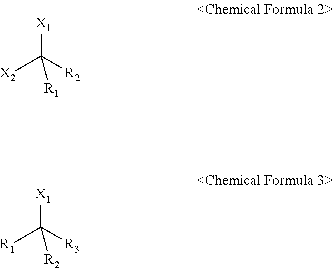

in <Chemical Formula 2> or <Chemical Formula 3>, X1 to X2 are independently hydrogen, chlorine element, and a chloroalkyl group having 1 to 5 carbon atoms,
R1 to R3 are independently selected from hydrogen, linear, branched, or cyclic alkyl groups having 1 to 5 carbon atoms, aryl groups having 6 to 12 carbon atoms, hydroxy groups having 0 to 4 carbon atoms, and alkoxy groups having 0 to 4 carbon atoms.

Specifically, the etching initiator may be any one of dichloromethyl methyl ether (DCMME), trimethyl chloro orthoacetate (TMCOA), and chloromethyl ethyl ether (CMEE).

Thereafter, a purge gas (for example, an inert gas such as Ar) is supplied to the inside of the chamber to discharge the unadsorbed etching initiator or by-products.

Thereafter, the substrate is exposed to a reactant (or reactive gas) supplied to the inside of the chamber, and etching is activated by the reactant. The reactant may be any one of O₃, O₂, H₂O, NH₃, H₂O, or H₂.

Thereafter, a purge gas (for example, an inert gas such as Ar) is supplied to the inside of the chamber to discharge the unreacted materials or by-products.

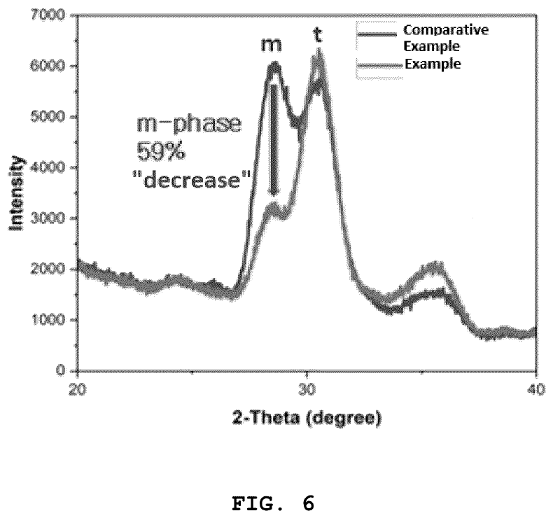

FIG. 5 is a diagram illustrating annealing/etching according to an embodiment of the present invention. FIG. 6 is XRD results of HfO₂ for Comparative Example 1/Example 1, FIG. 7 is TEM photos showing annealing/etching results according to Example 1.

### Comparative Example 1

1) An HfO₂ thin film with a thickness of 60 Å is prepared by ALD on an Si substrate.

2) Annealing is performed at 500° C. in an O₂ atmosphere.

### Example 1

1) HfO₂ thin film with a thickness of 60 Å is prepared by ALD on an Si substrate.

2) Nb₂O₅ stressor layer of 30 Å is deposited onto the HfO₂ film.

3) Annealing is performed at 500° C. in an O₂ atmosphere.

4) Trimethyl Orthoformate (TMOF) is adsorbed as the etch initiator, and then O₃ is supplied as the reactive gas to activate etching and remove the Nb₂O₅ stressor layer.

In Comparative Example 1, XRD analysis showed that a monoclinic/tetragonal peak height ratio of HfO₂ is 1:0.98. In contrast, XRD analysis of Example 1 showed a 59% decrease in monoclinic and 8% decrease in tetragonal peak heights, thereby improving the monoclinic/tetragonal peak height ratio to 1:2.58. In Example 1, the Nb₂O₅ stressor layer was confirmed to be removed.

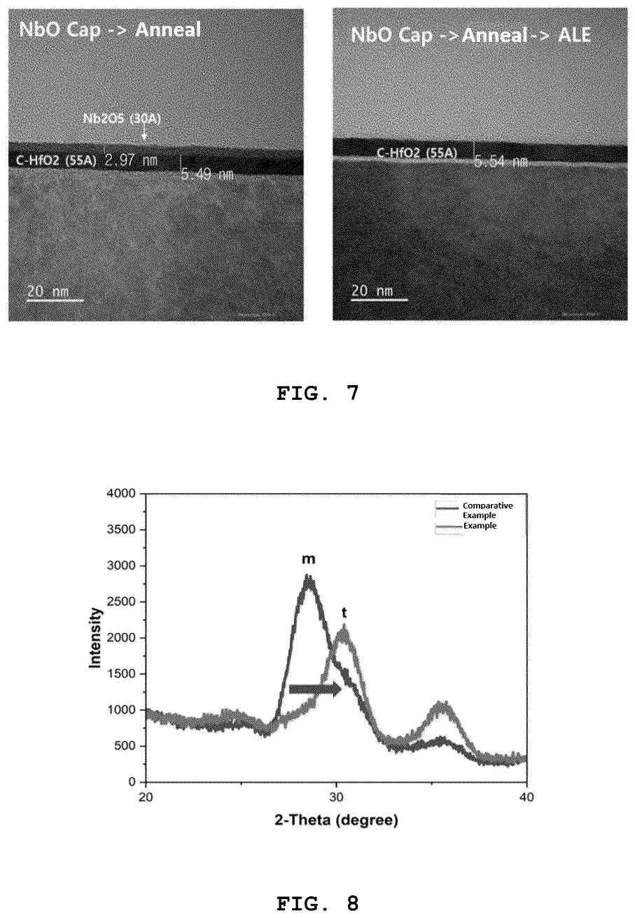

FIG. 8 is a graph showing the crystal structure of HfO₂ for Comparative Example 2/Example 2.

### Comparative Example 2

1) An HfO₂ thin film with a thickness of 40 Å is prepared by ALD on an Si substrate.

2) Annealing is performed at 500° C. in an O₂ atmosphere.

### Example 2

1) HfO₂ thin film with a thickness of 40 Å is prepared by ALD on an Si substrate.

2) Nb₂O₅ stressor layer of 30 Å is deposited onto the HfO₂ film.

3) Annealing is performed at 500° C. in an O₂ atmosphere.

4) Dimethylformamide dimethyl acetal (DFDA) is adsorbed as the etch initiator, and then O₃ is supplied as the reactive gas to activate etching and remove the Nb₂O₅ stressor layer.

In Comparative Example 2, XRD analysis showed the crystal structure of HfO₂ was monoclinic. In Example 2, the crystal structure of HfO₂ improved to tetragonal.

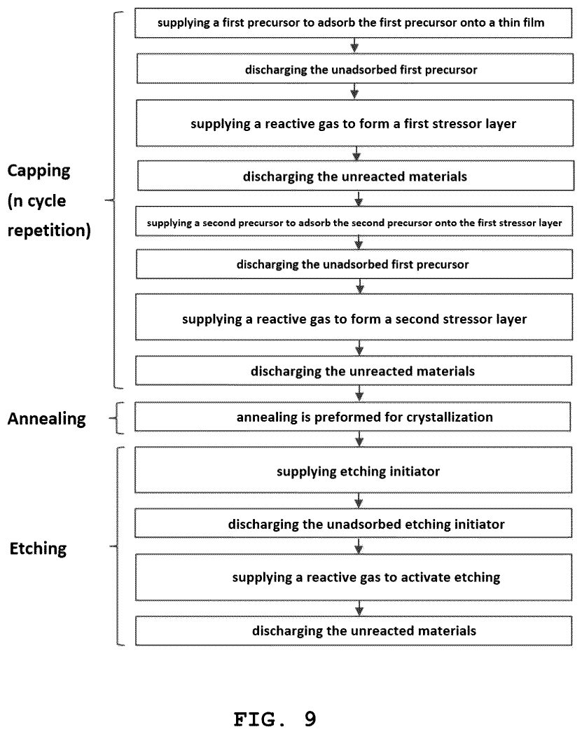

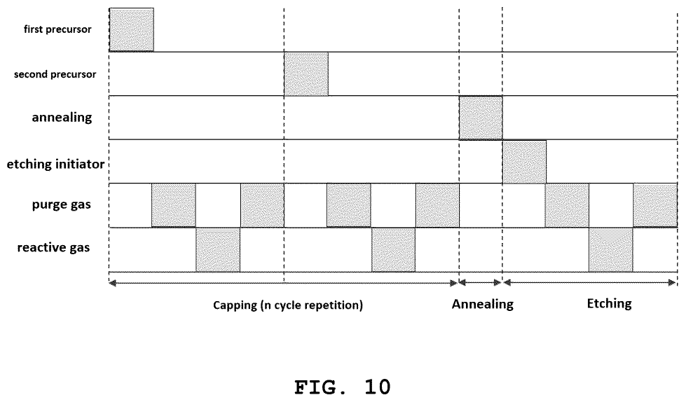

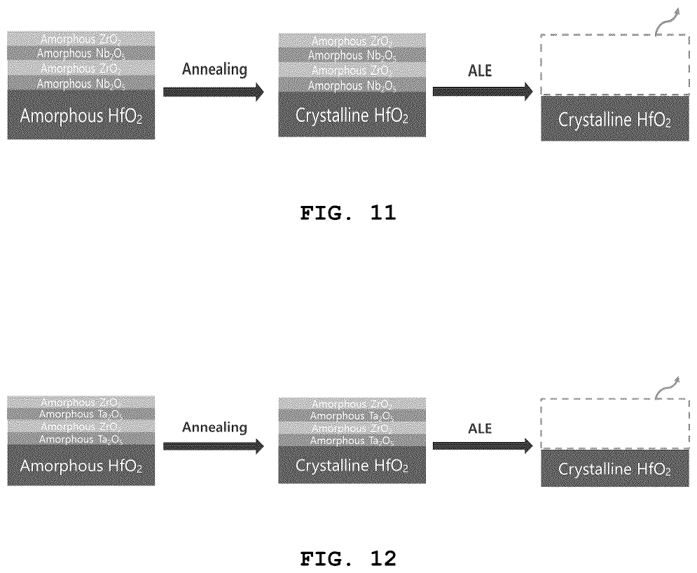

FIG. 9 is a flowchart illustrating a method of treating thin films according to another embodiment of the present invention, FIG. 10 is a graph schematically showing a supply cycle according to another embodiment of the present invention. FIGS. 11 and 12 are diagrams of annealing/etching according to an embodiment of the present invention.

The stressor layer can also be formed by mixing two or more thin films, including an element that assists in crystallization of the lower thin film. For example, the stressor layer may have one or more of Nb, Ta, Cr, Zr, Ru, Mo, or Sn as a central element. As shown in FIG. 9 and FIG. 10, the capping process can be repeated using two or more capping precursors.

In such cases, the mixed metal oxide layer can include one or more films capable of forming the same crystal as the lower thin film, thereby allowing the lower thin film to grow in the same crystal structure due to similar lattice constants, making it easier to form the desired crystal structure than capping with a single metal oxide. When two or more metal oxides are capped, the mixed layer is alternately stacked in thin sheets and remains amorphous after annealing, making it easy to remove the capping layer via etching (see FIGS. 11 and 12).

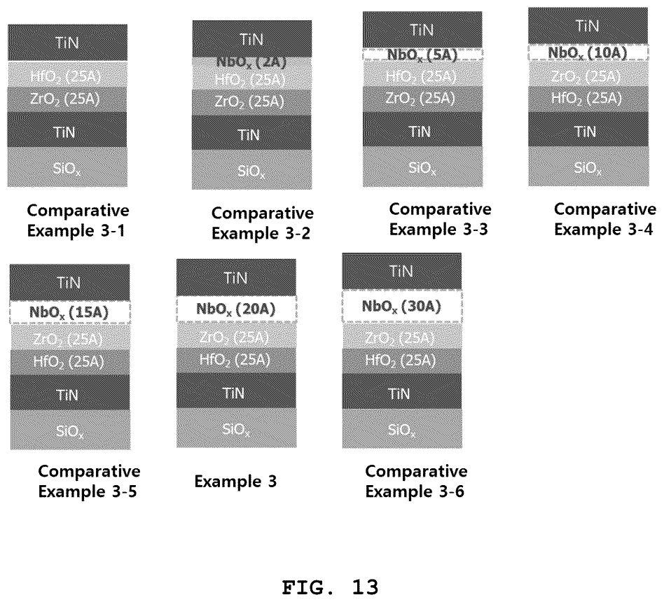

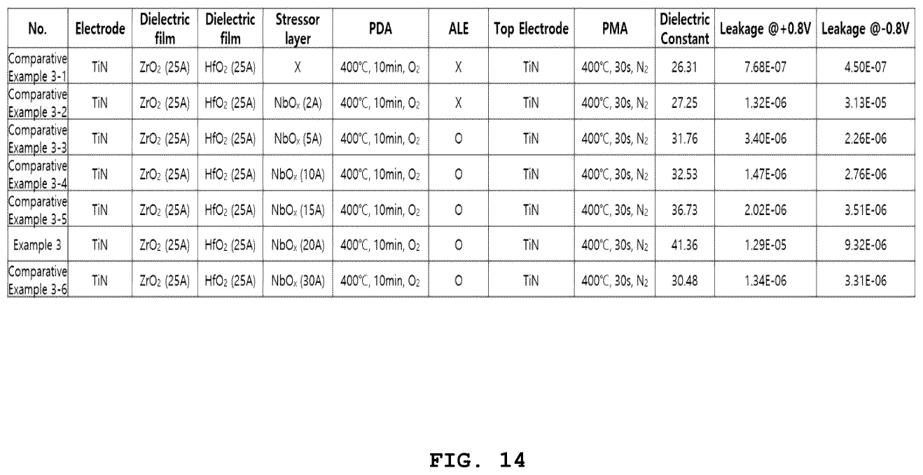

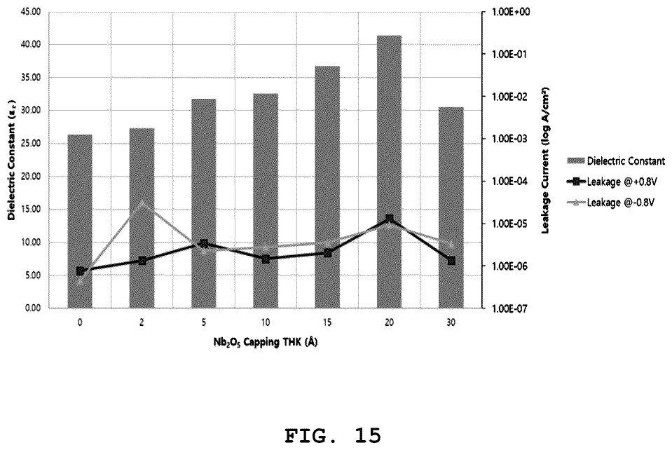

FIG. 13 is a diagram illustrating Comparative Examples 3-1 to 3-6, and Example 3, FIG. 14 is a table showing the electrical characteristics according to Comparative Examples 3-1 to 3-6, and Example 3. FIG. 15 is a graph showing the electrical characteristics according to Comparative Examples 3-1 to 3-6, and Example 3.

### Comparative Example 3-1

1) ZrO₂ thin film with a thickness of 25 Å is prepared by ALD on SiO/TiN substrate, followed by HfO₂ thin film with a thickness of 25 Å.

2) Annealing is performed at 400° C. for 10 min in an O₂ atmosphere.

3) Top electrode TiN is deposited, followed by annealing at 400° C. for 30 seconds in N₂ atmosphere.

Dielectric constant and leakage current measured as 26.31, 7.68E-07 @+0.8V, 4.50E-07 @−0.8V (see FIGS. 13, 14).

### Comparative Example 3-2

1) ZrO₂ thin film with a thickness of 25 Å is prepared by ALD on SiO/TiN substrate, followed by HfO₂ thin film with a thickness of 25 Å.

2) Nb₂O₅ stressor layer of 2 Å is deposited onto the HfO₂ film.

3) Annealing is performed at 400° C. for 10 min in an O₂ atmosphere.

4) Top electrode TiN is deposited, followed by annealing at 400° C. for 30 seconds in N₂ atmosphere.

Dielectric constant and leakage current measured as 27.25, 1.32E-06 @+0.8V, 3.13E-05 @−0.8V (see FIGS. 13, 14).

### Comparative Example 3-3

1) ZrO₂ thin film with a thickness of 25 Å is prepared by ALD on SiO/TiN substrate, followed by HfO₂ thin film with a thickness of 25 Å.

2) Nb₂O₅ stressor layer of 5 Å is deposited onto the HfO₂ film.

3) Annealing is performed at 400° C. for 10 min in an O₂ atmosphere.

4) Dimethylformamide dimethyl acetal (DFDA) is adsorbed as the etch initiator, and then O₃ is supplied as the reactive gas to activate etching and remove the Nb₂O₅ stressor layer.

5) Top electrode TiN is deposited, followed by annealing at 400° C. for 30 seconds in N₂ atmosphere.

Dielectric constant and leakage current measured as 31.76, 3.40E-06 @+0.8V, 2.26E-06 @−0.8V (see FIGS. 13, 14).

### Comparative Example 3-4

1) ZrO₂ thin film with a thickness of 25 Å is prepared by ALD on SiO/TiN substrate, followed by HfO₂ thin film with a thickness of 25 Å.

2) Nb₂O₅ stressor layer of 10 Å is deposited onto the HfO₂ film.

3) Annealing is performed at 400° C. for 10 min in an O₂ atmosphere.

4) Dimethylformamide dimethyl acetal (DFDA) is adsorbed as the etch initiator, and then O₃ is supplied as the reactive gas to activate etching and remove the Nb₂O₅ stressor layer.

5) Top electrode TiN is deposited, followed by annealing at 400° C. for 30 seconds in N₂ atmosphere.

Dielectric constant and leakage current measured as 32.53, 1.47E-06 @+0.8V, 2.76E-06 @−0.8V (see FIGS. 13, 14).

### Comparative Example 3-5

1) ZrO₂ thin film with a thickness of 25 Å is prepared by ALD on SiO/TiN substrate, followed by HfO₂ thin film with a thickness of 25 Å.

2) Nb₂O₅ stressor layer of 15 Å is deposited onto the HfO₂ film.

3) Annealing is performed at 400° C. for 10 min in an O₂ atmosphere.

4) Dimethylformamide dimethyl acetal (DFDA) is adsorbed as the etch initiator, and then O₃ is supplied as the reactive gas to activate etching and remove the Nb₂O₅ stressor layer.

5) Top electrode TiN is deposited, followed by annealing at 400° C. for 30 seconds in N₂ atmosphere.

Dielectric constant and leakage current measured as 36.73, 2.02E-06 @+0.8V, 3.51E-06 @−0.8V (see FIGS. 13, 14).

### Comparative Example 3-6

1) ZrO₂ thin film with a thickness of 25 Å is prepared by ALD on SiO/TiN substrate, followed by HfO₂ thin film with a thickness of 25 Å.

2) Nb₂O₅ stressor layer of 30 Å is deposited onto the HfO₂ film.

3) Annealing is performed at 400° C. for 10 min in an O₂ atmosphere.

4) Dimethylformamide dimethyl acetal (DFDA) is adsorbed as the etch initiator, and then O₃ is supplied as the reactive gas to activate etching and remove the Nb₂O₅ stressor layer.

5) Top electrode TiN is deposited, followed by annealing at 400° C. for 30 seconds in N₂ atmosphere.

Dielectric constant and leakage current measured as 30.48, 1.34E-06 @+0.8V, 3.31E-06 @−0.8V (see FIGS. 13, 14).

### Example 3

1) ZrO₂ thin film with a thickness of 25 Å is prepared by ALD on SiO/TiN substrate, followed by HfO₂ thin film with a thickness of 25 Å.

2) Nb₂O₅ stressor layer of 20 Å is deposited onto the HfO₂ film.

3) Annealing is performed at 400° C. for 10 min in an O₂ atmosphere.

4) Dimethylformamide dimethyl acetal (DFDA) is adsorbed as the etch initiator, and then O₃ is supplied as the reactive gas to activate etching and remove the Nb₂O₅ stressor layer.

5) Top electrode TiN is deposited, followed by annealing at 400° C. for 30 seconds in N₂ atmosphere.

Dielectric constant and leakage current measured as 41.36, 1.29E-05 @+0.8V, 9.32E-06 @−0.8V (see FIGS. 13, 14).

From these results, in a composite film with a ZrO₂/HfO₂ ratio of 1:1, an Nb₂O₅ capping thickness of 20 Å provided the largest dielectric constant enhancement with leakage current levels similar to or improved over controls (see FIG. 15). This improvement in dielectric constant increases device capacitance and allows low voltage operation, thus improving device performance and integration.

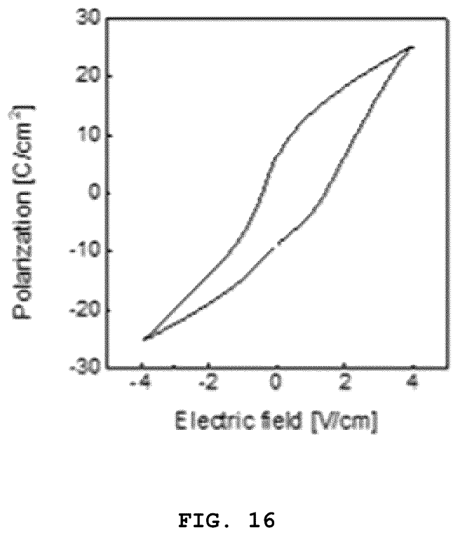

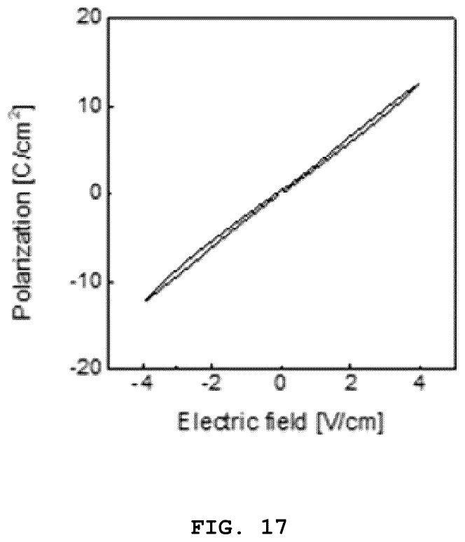

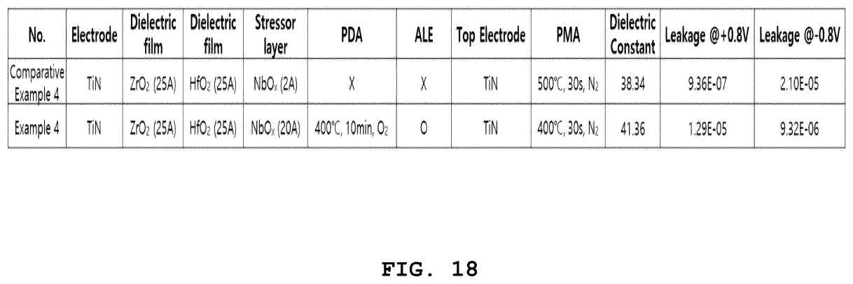

FIG. 16 is a graph showing P-E curve for the thin film of Comparative Example 4, FIG. 17 is a graph showing P-E curve for the thin film of Example 4. FIG. 18 is a table showing the electrical characteristics according to Comparative Example 4/Example 4.

### Comparative Example 4

1) ZrO₂ thin film with a thickness of 25 Å is prepared by ALD on SiO/TiN substrate, followed by HfO₂ thin film with a thickness of 25 Å.

2) Nb₂O₅ stressor layer of 2 Å is deposited onto the HfO₂ film.

3) Top electrode TiN is deposited, followed by annealing at 500° C. for 30 seconds in N₂ atmosphere.

Leakage current measured as 9.36E-07@+0.8V, 2.10E-05@−0.8V (see FIG. 18). Also, in P-E curve, 2Pr value was 15.15, indicating ferroelectric characteristic (see FIG. 16).

### Example 4

1) ZrO₂ thin film with a thickness of 25 Å is prepared by ALD on SiO/TiN substrate, followed by HfO₂ thin film with a thickness of 25 Å.

2) Nb₂O₅ stressor layer of 30 Å is deposited onto the HfO₂ film.

3) Annealing is performed at 400° C. for 10 min in an O₂ atmosphere.

4) Dimethylformamide dimethyl acetal (DFDA) is adsorbed as the etch initiator, and then O₃ is supplied as the reactive gas to activate etching and remove the Nb₂O₅ stressor layer.

5) Top electrode TiN is deposited, followed by annealing at 500° C. for 30 seconds in N₂ atmosphere.

Leakage current measured as 1.91E-06@+0.8V, 2.34E-06@−0.8V (see FIG. 18). Also, in P-E curve, no ferroelectric characteristic was observed (see FIG. 17, FIG. 18).

Thus, ferroelectric characteristic can be controlled by appropriately adjusting capping and annealing conditions, thereby improving operating voltage, reliability, and durability of the memory device.

When a dielectric film is formed with a high-k material, it is difficult to form a desired crystal structure at a low thickness, so the desired crystal structure in the dielectric film can be achieved by capping the dielectric film with another metal oxide. In this case, since some of the capping layer may remain and deteriorate device properties, the capping layer thickness is limited. Therefore, it is difficult to form the capping layer of sufficient thickness to obtain the desired crystal structure in the dielectric film. However, the present invention allows the capping layer of sufficient thickness, as the capping layer can be removed by subsequent atomic layer etching (ALE), thus obtaining the desired crystal structure in the dielectric film.

The present invention has been explained in detail with reference to embodiments, but other embodiments may be included. Accordingly, the technical idea and scope described in the claims below are not limited to the embodiments.

1. A method of treating thin films, the method comprising:
supplying a capping precursor to the inside of a chamber where a substrate is placed to adsorb the capping precursor onto a thin film formed on the substrate;
purging the inside of the chamber;
supplying a first reaction material to the inside of the chamber to form a stressor layer;
purging the inside of the chamber;
annealing the substrate;
supplying an etch initiator to the inside of the chamber;
purging the inside of the chamber;
supplying a second reaction material to the inside of the chamber to activate the etch initiator; and
purging the inside of the chamber.

2. A method of treating thin films, the method comprising:
forming first and second stressor layers on a thin film formed on a substrate, alternately and repeatedly;
annealing the substrate;
supplying an etch initiator to the inside of the chamber where the substrate is placed;
purging the inside of the chamber;
supplying a third reaction material to the inside of the chamber to activate the etch initiator; and
purging the inside of the chamber,
wherein forming the first stressor layer comprising:
supplying a first capping precursor to the inside of a chamber to adsorb the first capping precursor onto the thin film;
purging the inside of the chamber;
supplying a first reaction material to the inside of the chamber to form a first stressor layer;
purging the inside of the chamber,
wherein forming the second stressor layer comprising:
supplying a second capping precursor to the inside of a chamber to adsorb the second capping precursor onto the thin film;
purging the inside of the chamber;
supplying a second reaction material to the inside of the chamber to form a second stressor layer;
purging the inside of the chamber.

3. The method of claim 1, wherein the etching initiator is represented by the following <Chemical Formula 1>: 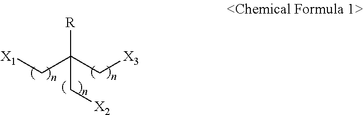
in <Chemical Formula 1>, n is each independently selected from integers of 0 to 5,
X1 to X3 are each independently selected from an alkoxy group having 1 to 5 carbon atoms, and a dialkylamino group having 1 to 5 carbon atoms, and
R is selected from hydrogen, a linear, branched, or cyclic alkyl groups having 1 to 5 carbon atoms, an alkoxy group having 1 to 5 carbon atoms, and a dialkylamino group having 1 to 5 carbon atoms.

4. The method of claim 3, wherein the etching initiator is any one of Trimethyl orthoformate (TMOF), Triethyl orthoformate (TEOF), Dimethylformamide dimethyl acetal (DFDA), and Tris(dimethylamino)methane (TDMAM).

5. The method of claim 1, wherein the etching initiator is represented by the following <Chemical Formula 2> or <Chemical Formula 3>: 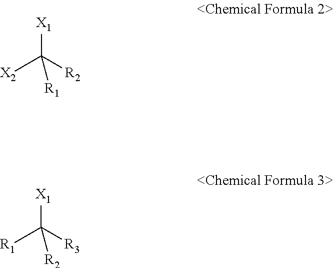
in <Chemical Formula 2> or <Chemical Formula 3>, X1 to X2 are independently selected from hydrogen, chlorine element, and a chloroalkyl group having 1 to 5 carbon atoms,
R1 to R3 are independently selected from hydrogen, linear, branched, or cyclic alkyl groups having 1 to 5 carbon atoms, aryl groups having 6 to 12 carbon atoms, hydroxy groups having 0 to 4 carbon atoms, or alkoxy groups having 0 to 4 carbon atoms.

6. The method of claim 1, wherein the etching initiator is any one of dichloromethyl methyl ether (DCMME), trimethyl chloro orthoacetate (TMCOA), and chloromethyl ethyl ether (CMEE).

7. The method of claim 1, wherein the thin film is a metal oxide film having one of Al, Nb, Hf, Ti, Si, Ta, Mo, Zr, and W as a central element.

8. The method of claim 1, wherein the stressor layer is a metal oxide film having one of Nb, Ta, Cr, Zr, Ru, Mo, and Sn as a central element.

9. The method of claim 1, wherein the stressor layer has a band gap smaller than that of the thin film.

10. The method of claim 1, wherein the stressor layer has a higher crystallization temperature than the thin film.

11. The method of claim 1, wherein the reactant is any one of O₃, O₂, or H₂O.

12. The method of claim 1, which is performed at 50 to 700° C.

13. The method of claim 1, wherein the thin film and the stressor layer are any one of a metal film, a metal oxide, a metal nitride, a metal sulfide, a silicon nitride, and a silicon oxide.

14. The method of claim 1, wherein the thin film is a binary compound or a ternary compound doped with one or more elements.

15. A method of manufacturing a memory device, the method comprising the method of treating thin films according to claim 1.

16. The method of claim 2, wherein the etching initiator is represented by the following <Chemical Formula 1>: 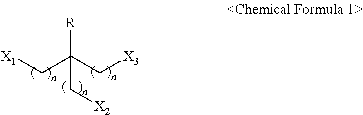
in <Chemical Formula 1>, n is each independently selected from integers of 0 to 5,
X1 to X3 are each independently selected from an alkoxy group having 1 to 5 carbon atoms, and a dialkylamino group having 1 to 5 carbon atoms, and
R is selected from hydrogen, a linear, branched, or cyclic alkyl groups having 1 to 5 carbon atoms, an alkoxy group having 1 to 5 carbon atoms, and a dialkylamino group having 1 to 5 carbon atoms.

17. The method of claim 2, wherein the etching initiator is represented by the following <Chemical Formula 2> or <Chemical Formula 3>: 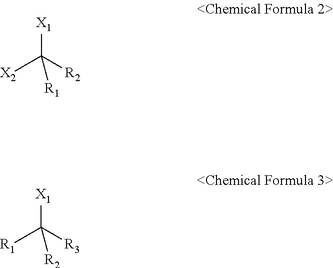
in <Chemical Formula 2> or <Chemical Formula 3>, X1 to X2 are independently selected from hydrogen, chlorine element, and a chloroalkyl group having 1 to 5 carbon atoms,
R1 to R3 are independently selected from hydrogen, linear, branched, or cyclic alkyl groups having 1 to 5 carbon atoms, aryl groups having 6 to 12 carbon atoms, hydroxy groups having 0 to 4 carbon atoms, or alkoxy groups having 0 to 4 carbon atoms.

18. The method of claim 2, wherein the thin film is a metal oxide film having one of Al, Nb, Hf, Ti, Si, Ta, Mo, Zr, and W as a central element.

19. The method of claim 2, wherein the stressor layer is a metal oxide film having one of Nb, Ta, Cr, Zr, Ru, Mo, and Sn as a central element.

20. The method of claim 2, wherein the stressor layer has a band gap smaller than that of the thin film.
